(19) United States
(12) Patent Application Publication
(10) Pub. No.: US 2009/0204189 A1
(43) Pub. Date: Aug. 13, 2009

(54) Living-Tissue Normalization Method

(75) Inventors: Yuhei Kurata, Kumamoto (JP); Hidetoshi Furushima, Kumamoto (JP); Hirohumi Kai, Kumamoto (JP)

Correspondence Address: RADER FISHMAN & GRAUER PLLC, LION BUILDING, 1233 20TH STREET N.W., SUITE 501, WASHINGTON, DC, 20036, US

(73) Assignee: Tuchiyagomu Co., Ltd., Kumamoto (JP)

(21) Appl. No.: 12/226,016

(22) PCT Filed: Apr. 5, 2006
(86) PCT No.: PCT/JP2006/307239

§ 371 Date: Mar. 17, 2009

(51) Int. Cl. A61F 7/08; A61K 38/16

(52) U.S. Cl. 607/99; 514/12

(57) ABSTRACT

A living-tissue normalization method and therapeutic device. The method includes intermittently flowing a slight direct current through a living body or living tissue at predetermined regular intervals to thereby activate a normalization mechanism of said living body or living tissue through the intermediary of protein. The therapeutic device includes a resistance provided between a pair of electrodes and current control arranged so that electrically conductive layers of a pair of pad elements are intermittently supplied with a slight direct current at predetermined regular intervals, and so that the pair of electrodes are supplied with a direct current.

### TECHNICAL FIELD

The present invention relates to a living-tissue normalization method for activating a living body or a living tissue, and more particularly relates to such a living-tissue normalization method for activating a normalization mechanism for a living body or a living tissue.

### BACKGROUND OF ART

Recently, a function of ubiquitin existing in all cells has been analyzed. Ubiquitin is combined with needless protein, and serves as a mark of the needless protein. The needless protein with which the ubiquitin is combined (ubiquitinated protein) is took in enzyme, i.e., proteasome by using the ubiquitin as the mark so as to be resolved. This living-body normalization mechanism, in which the needless protein is resolved, functions as a ubiquitin-proteasome system, and is concerned with tissue normalization regarding division of cells, repair of DNA, quality control of protein, immunity and so on.

Also, in the living-body normalization mechanism, a given part of a living body is warmed by using an electric warming type therapeutic device so as to express and induce a heat shock protein (which is referred to as an HASP hereinafter), and thus is activated.

The HSP is called a stress protein, and represents a group of proteins having a molecular weight falling within a range between several tens of thousands and about 150,000, with the proteins being sorted into several families in accordance with the molecular weights thereof. The HSP is non-covalently bonded to hydrophobic regions of a renewal protein, a denatured protein and an abnormal protein, to thereby assist a folding of proteins, a transportation into a cellular organelle and a re-folding and resolution of denatured proteins, and thus a quality control of intracellular proteins is carried out to thereby prevent accumulation of abnormal proteins and denatured proteins in cells. These functions are generally named a molecular chaperone, and the HSP is induced by not only a heat shock but also a variety of physical and chemical injury factors. It is the established fact that a cell, in which a large amount of HSP is expressed, acquires a strong resistance to a variety of injury factors.

HSP70 having the molecular weight of 72 kDa, which is belonged to an HSP70 family, is a protein initially induced by a stress, and research of this protein is most advanced. When an excess of HSP70 is expressed by exposing cells to a nonfatal stress such as a heat shock or the like, the cells exhibits a strong resistance against fatal injury factors for survival.

It is known that this resistance not only prevents accumulation of abnormal proteins and denatured proteins in cells through a function of the molecular chaperone, but also preserves a cellular organelle such as a mitochondrion in a cell subjected to a stress, to thereby suppress cellular necrosis, inflammable reaction and apoptosis, resulting in reduction of cell loss (see: Samali, A. et al., Cell Stress & Chaperones 3:228, 1998).

In a variety of diseased conditions, cells are exposed to physical and chemical stresses. In many animal models of human diseases using laboratory animals, it is disclosed that an injury is reduced when an excess of HSP70 is expressed by certain methods, a clinical application regarding HSP70 is increasingly expected (see: Minowada, G. et al. J. Clin, Invest., 95:2, 1995, and Documents Cited Therein).

Referring to a variety of stresses to which cells are subjected with relation to human diseases, first, there is an ischemia as a typical stress. In a case where an excess of HSP70 is previously expressed in laboratory animals by subjecting them to a heat shock load all over, it is known that infarted regions of a brain (Kitagawa, K. et al., J. Cereb. Blood Flow Metab., 11:449, 1991) and a heat (Donnelly, T. J. et al., Circulation 85:1048, 1992) can be reduced even if a cerebral artery and coronary artery are ligated. Also, in a mouse into which a gene of HSP70 is introduced, it is known that a cardiac infarction can be suppressed (Maber, M. S. et al., J. Clin. Invest., 95:1446, 1995). Suppression of cellular injuries by the ischemia of HSP70 are applicable to not only a brain and a heart but also all organs.

An production amount of active-oxygen and free radical is increased due to infection diseases, inflammations, degenerative diseases, autoimmune diseases, arteriosclerosis and aging, to thereby cause cellular injuries. It is known that HSP70 can suppress the cellular injuries caused by the active-oxygen and free radical (Polla B. S. et al., Proc. Natl. Acad Sci. USA, 93:6458, 1996).

It is established that one of major factors causing an ischemia/reperfusion injury is a facilitation of production of active-oxygen during reperfusion, and it is known that the ischemia/reperfusion injury can be reduced by HSP70 in a brain, a heart, a liver, a small intestine and so on. This function obtained by HSP70 are applicable to all organs. Also, an organ plant is a typical example representing the ischemia/reperfusion injury. In reality, it is known that a take rate in grafting a piece of skin can be improved by expressing an excess of HSP70 (Koenig, W. J. et al., Plast Recontsr. Surg., 90:659, 1992), and it is reported that acute rejection in transplant of a liver can be more reduced as expression of HSP70 become larger (Flohe, S. et al., Traspl. Int., 11:89, 1998). Also, ultraviolet rays, radiation rays, heavy metals, alcohol, anticancer drugs or paraquat causes an injury which is mainly based on the active-oxygen and free radical. HSP70 can be expected to be effective in not only prevention and treatment of injuries on skins, mucous membranes, eye lenses and retinas but also treatment of alcoholic organ injuries and treatment of heavy metal and chemical poisoning.

Further, since cancer cells express HSP70 on the surfaces of the cells so that NK cells are activated by HSP70 (Kurosawa, S. et al., Eur. J. Immunol., 23:1029, 1993), it is possible to activate immunity against the cancer through the expression of HSP70. Also, it is known that, upon invasion of microorganisms, resistance to infection is more increased as expression of HSP of host macrophage is stronger (Denagel, D. C. et al., Crit. Rev. Immunol., 13:71, 1993), and thus it is possible to expect an increase in activation of the immunity and defective ability of a living body.

When it is noticed that HSP70 has a function of a quality control of intracellular proteins, HSP70 can be expected to be effective in diseases that abnormal proteins are accumulated in cells, such as Alzheimer's disease derived from accumulation of β-amyloid and Creutzfeldt-Jakob's disease derived from accumulation of abnormal prion, and degenerative diseases, such as amyloidosis, Wilson's disease, Parkinson's disease and so on.

Also, not only can HSP70 be expected to be effective in resistance to a physical stress, such as a surgical operation, a surface wound, an living body invasion and so on, but also it can be expected to suppress crisis and exacerbation of allergy diseases, stress cankers chronic inflammation diseases and so on, which are derived from metal stresses. Further, it is reported that HSP70 can effective in mitigation of multiple-organ failures/shock derived from blood poisoning (Hauser, G. J. et al., Am. J. Physiol., 271:H2529, 1996), and improvement of a prognosis of adult respiratory distress syndrome (Villar, J. et al., Am. Rev. Respir. Dis., 147:177, 1993), and HSP70 can be expected as a curative medicine for serious symptoms of these living body inversions.

Since HSP is an intracellular (organic) substance, there is little possibility of causing side effects with an induction of HSP. Also, there is no report on diseases caused by an excess expression of HSP70. In animal testing, although a whole body heat shock loading, a temporary obstruction in a bloodstream, an HSP70 gene introduction and so on are carried out, it is difficult to apply these to an actual clinical trail. For these reasons, it can be said that a device, which is able to selectively induce HSP without injuring tissues and cells, is a clinically superior therapeutic device.

Since a transcription factor occupies the lowest position in a signal communication system from an external, it is considered that side effects can be minimally suppressed by utilizing that as a target. NF-κB is one of transcription factors, and is combined with IκB or inhibition proteins to hereby be inactivated. When cells are subjected to a variety of stimuli, the IκBi is phosphorylated, and then is ubiquitinated to thereby be resolved by proteasome. The loosed NF-κB is moved into a nucleus so that a variety of genes are specifically activated. Among genes which are under control of NF-κB, there is cytokine (TNF-α, β; IL-2, 6, 8 and so on) or the like which significantly works in cells of the immune system. Since these genes are expressed and induced when the cells are subjected to the stimuli, it is found that NF-κB is deeply concerned with an immune response. However, it is known that a variety of diseases are caused when its inflammation response is excessive. For example, since NF-κB is concerned with a variety of inflammatory diseases such as rheumatism, asthma, dermatitis and so on, and other diseases such as autoimmune disease, viral disease, arteriosclerosis and so on, control of NF-κB is clinically very significant (Anning Lin. Cancer Biology, 2003; Aggarwal B B et al. Indian J Exp Biol, 2004; and Alok C. Bharti et al. Biochemical Pharmacology, 2002).
Non-Patent Document 1: Samali, A. et al., Cell Stress & Chaperones 3:228, 1998
Non-Patent Document 2: Minowada, G. et al. J. Clin, Invest., 95:2, 1995, and Documents Cited Therein
Non-Patent Document 3: Kitagawa, K. et al., J. Cereb. Blood Flow Metab., 11:449, 1991
Non-Patent Document 4: Donnelly, T. J. et al., Circulation 85:1048, 1992
Non-Patent Document 5: Maber, M. S. et al., J. Clin. Invest., 95:1446, 1995
Non-Patent Document 6: Polla B. S. et al., Proc. Natl. Acad Sci. USA, 93:6458, 1996
Non-Patent Document 7: Koenig, W. J. et al., Plast Recontsr. Surg., 90:659, 1992
Non-Patent Document 8: Flohe, S. et al., Traspl. Int., 11:89, 1998
Non-Patent Document 9: Kurosawa, S. et al., Eur. J. Immunol., 23:1029, 1993
Non-Patent Document 10: Denagel, D. C. et al., Crit. Rev. Immunol., 13:71, 1993
Non-Patent Document 11: Hauser, G. J. et al., Am. J. Physiol., 271:H2529, 1996
Non-Patent Document 12: Villar, J. et al., Am. Rev. Respir. Dis., 147:177, 1993
Non-Patent Document 13: Anning Lin. Cancer Biology, 2003
Non-Patent Document 14: Aggarwal BB et al. Indian J Exp Biol, 2004
Non-Patent Document 15: Alok C. Bharti et al. Biochemical Pharmacology, 2002

### DISCLOSURE OF THE INVENTION

### Problems to be Resolved by the Invention

However, in the aforesaid living-body normalization mechanism based on the ubiquitin-proteasome system, there is a problem that needless proteins cannot be quickly and surely resolved and eliminated in that an amount of ubiquitin existing in all cells is not enough when living tissues are normal (health). Also, while living tissues are abnormal (disease), there is another problem that disorders or diseases cannot be improved in that needless proteins combined with ubiquitin are resolved and eliminated to thereby reduce ubiquitin in cells so that abnormal (disease) cells are not brought to programmed cell death whereby the abnormal cells such as the tumors or the like cannot be reduced.

On the other hand, in the living-body normalization mechanism based on HSP, using the electric warming type therapeutic device and so on, there is a problem that a living body cannot be quickly and accurately normalized, because HSP cannot be sufficiently expressed and induced, and thus cannot be sufficiently activated unless the living body is heated at a high temperature (e.g., 42° C.) over a time period of more than one hour so that a living tissue may be subjected to injury or the like due to the heating.

Further, there is no method of normalizing a living body by exerting an influence on an amount of IκB and a phosphorylation state which control activation of NF-κB.

The present invention has been developed to solve the above-mentioned problems, and an object of the present invention is to provide a living-tissue normalization method of quickly and accurately normalizing a living body or a living tissue.

### Means for Solving the Problems

In a living-tissue normalization method according to the present invention, a slight direct current is intermittently flowed through a living body or living tissue at predetermined regular intervals to thereby activate a normalization mechanism of the living body or living tissue through the intermediary of protein. Like this, according to the present invention, since the slight direct current is intermittently flowed through the living body or living tissue at the predetermined regular intervals to thereby activate the normalization mechanism of the living body or living tissue through the intermediary of protein, it is possible to quickly and accurately carry out normalization of an abnormal living body or living tissue.

Also, in the living-tissue normalization method according to the present invention, if necessary, heat is applied to the living body or living tissue through which the slight direct current is flowed. Like this, according to the present invention, since the heat is applied to the living body or living tissue through which the slight direct current is flowed, it is possible to more quickly and accurately carry out the normalization of the living body or living tissue.

Also, in the living-tissue normalization method according to the present invention, if necessary, the protein comprises ubiquitinated protein. Like this, according to the present invention, since the protein comprises the ubiquitinated protein, it is possible to more quickly and accurately carry out the normalization of the living body or living tissue.

Also, in the living-tissue normalization method according to the present invention, if necessary, the protein comprises heat shock protein. Like this, according to the present invention, since the protein comprises the heat shock protein, it is possible to more quickly and accurately carry out the normalization of the living body or living tissue.

Also, in the living-tissue normalization method according to the present invention, if necessary, the protein comprises I-κB protein. Like this, according to the present invention, since the protein comprises the I-κB protein, it is possible to more quickly and accurately carry out the normalization of the living body or living tissue.

Also, in the living-tissue normalization method according to the present invention, if necessary, the intermittent intervals of the slight direct current falls within a range from 30 Hz to 100 Hz. Like this, according to the present invention, since the intermittent intervals of the slight direct current falls within the range from 30 Hz to 100 Hz, it is possible to more quickly and accurately carry out the normalization of the living body or living tissue.

Also, in the living-tissue normalization method according to the present invention, if necessary, a temperature of the heating falls within a range from 38° C. to 45° C. Like this, according to the present invention, since the temperature of the heating falls within a range from 38° C. to 45° C., it is possible to more quickly and accurately carry out the normalization of the living body or living tissue.

In a therapeutic device according to the present invention: a pair of pad elements are adhered to different parts of a living body or living tissue; an electrical current is flowed between the pair of pad elements through a current control means to thereby cure the living body; each of the pad elements includes an electrically conductive layer adhered to a surface of the living body or living tissue and comprising an electrically conductive sheet, an electrically insulative layer provided on a rear face of the electrically conductive layer and comprising an electrically insulative sheet exhibiting a thermally conductive characteristic, and a heating layer provided on a rear face of the electrically insulative layer and comprising a pair of electrodes arranged at the side ends thereof and a resistance provided between the pair of electrodes; and the current control means is arranged so that the electrically conductive layers of the pair of pad elements are intermittently supplied with a slight direct current at predetermined regular intervals, and so that the pair of electrodes are supplied with a direct current.

like this, according to the present invention, since the pair of pad elements are adhered to the different parts of the living body or living tissue; since the electrical current is flowed between the pair of pad elements through the current control means to thereby cure the living body; since each of the pad elements includes the electrically conductive layer adhered to the surface of the living body or living tissue and comprising the electrically conductive sheet, the electrically insulative layer provided on the rear face of the electrically conductive layer and comprising the electrically insulative sheet exhibiting the thermally conductive characteristic, and the heating layer provided on the rear face of the electrically insulative layer and comprising the pair of electrodes arranged at the side ends thereof and the resistance provided between the pair of electrodes; and since the current control means is arranged so that the electrically conductive layers of the pair of pad elements are intermittently supplied with the slight direct current at the predetermined regular intervals, and so that the pair of electrodes are supplied with the direct current, the living body or living tissue, through which the slight direct current is flowed, can be simultaneously heated so that it is possible to quickly and accurately carry out normalization of a living body or living tissue.

Also, in the therapeutic device according to the present invention, if necessary, each of the pair of electrodes is shaped as a strip-like electrode in the heating layer of the pad element, and the resistance is formed of a plurality of carbon fibers extending in parallel to the pair of electrodes therebetween. Like this, according to the present invention, since each of the pair of electrodes is shaped as the strip-like electrode in the heating layer of the pad element, and since the resistance is formed of the plurality of carbon fibers extending in parallel to the pair of electrodes therebetween, the heating can be carried out without causing short circuits between the pair of electrodes, due to suitable resistance values between the carbon fibers extending in parallel to each other, so that it is possible to more quickly and accurately carry out the normalization of the living body or living tissue.

Also, in the therapeutic device according to the present invention, if necessary, the direct current flowing into each of the electrically conductive layers is intermittently supplied at a frequency falling within a range from 50 Hz to 60 Hz. Like this, according to the present invention, since the direct current flowing into each of the electrically conductive layers is intermittently supplied at the frequency falling within the range from 50 Hz to 60 Hz, the living body or living tissue can be effectively subjected to current stimuli, so that it is possible to more quickly and accurately carry out the normalization of the living body or living tissue.

Also, in the therapeutic device according to the present invention, if necessary, the heating layer of each of the pair of pad elements is heated at a temperature falling within a range from 38° C. to 45° C. Like this, according to the present invention, since the heating layer of each of the pair of pad elements is heated at the temperature falling within the range from 38° C. to 45° C., the living body or living tissue can be suitably subjected to thermal stimuli, so that it is possible to more quickly and accurately carry out the normalization of the living body or living tissue.

Also, in the therapeutic device according to the present invention, if necessary, the direct current flowing through the electrically conductive layer features a supply time of at least 1 min., and the direct current has a voltage which is in reverse proportion to the supply time, and which is set within a range from 0.01 V to 0.4 V. Like this, according to the present invention, since the direct current flowing through the electrically conductive layer features the supply time of at least 1 min., and since the direct current has the voltage which is in reverse proportion to the supply time, and which is set within the range from 0.03 V to 0.2 V, the current stimuli and the thermal stimuli can be suitably balanced with each other, so that it is possible to more quickly and accurately carry out the normalization of the living body or living tissue.

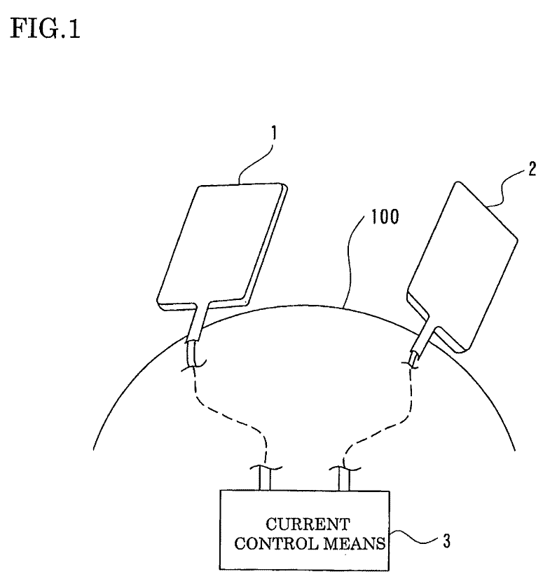

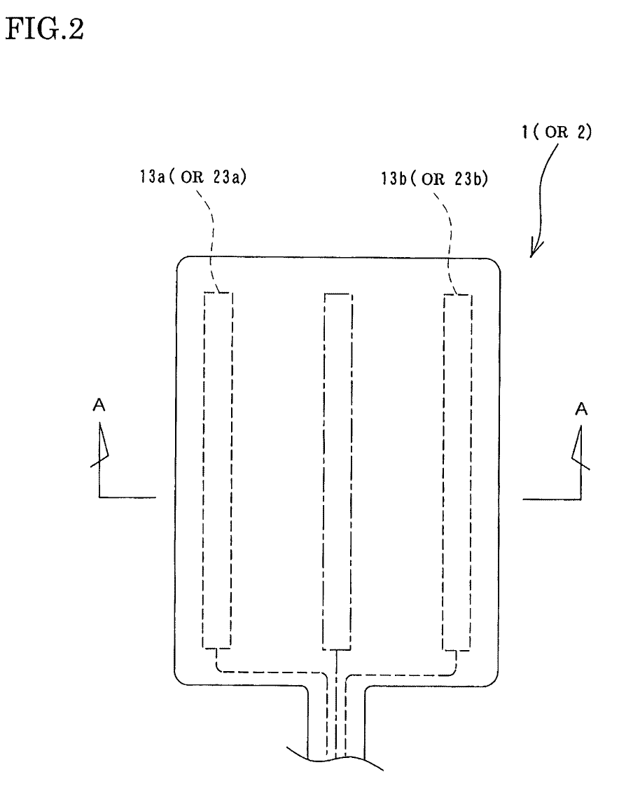

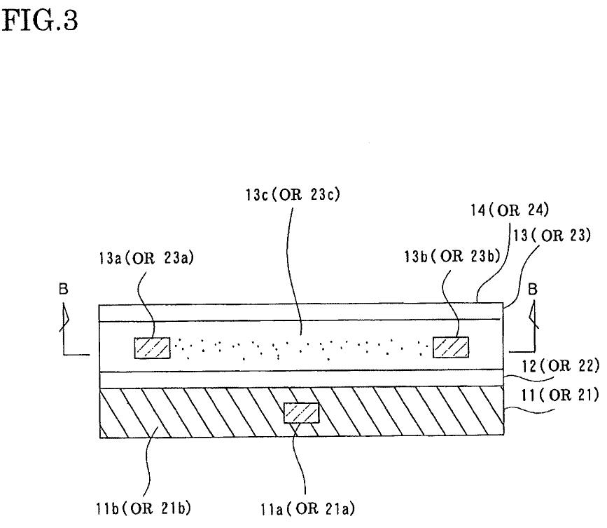

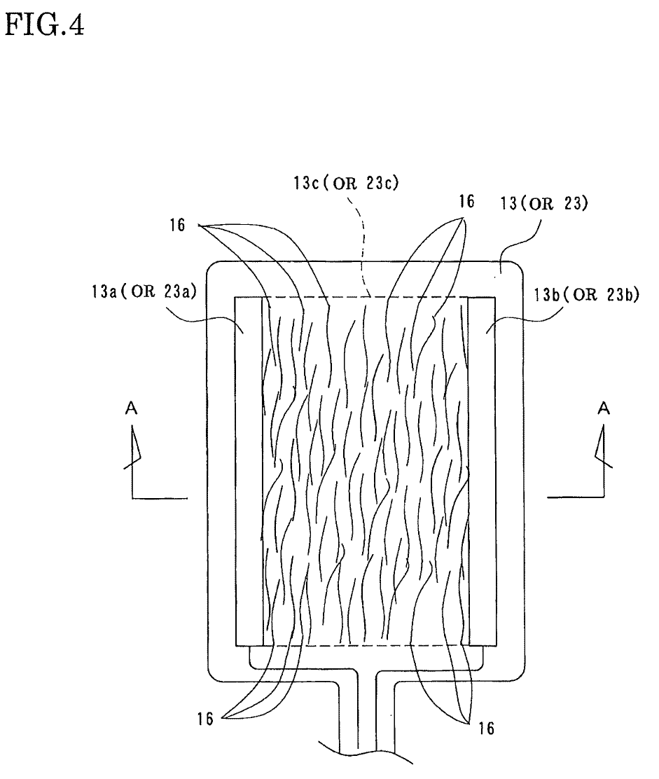

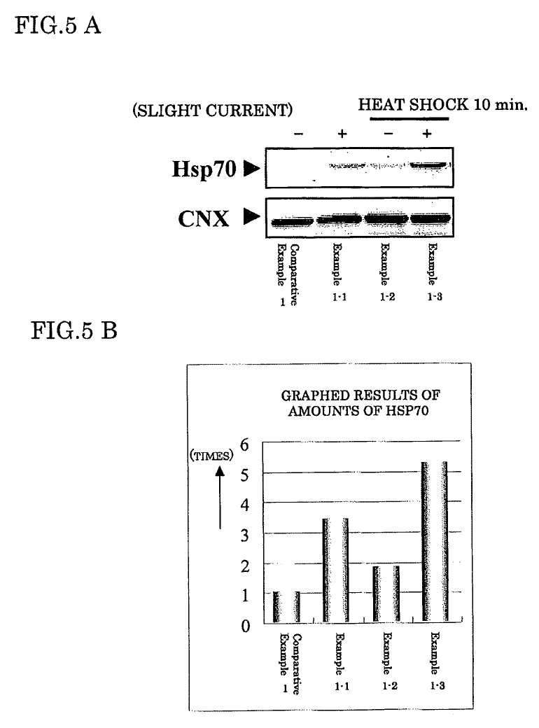

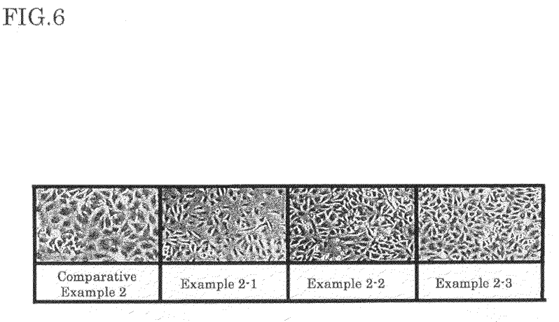

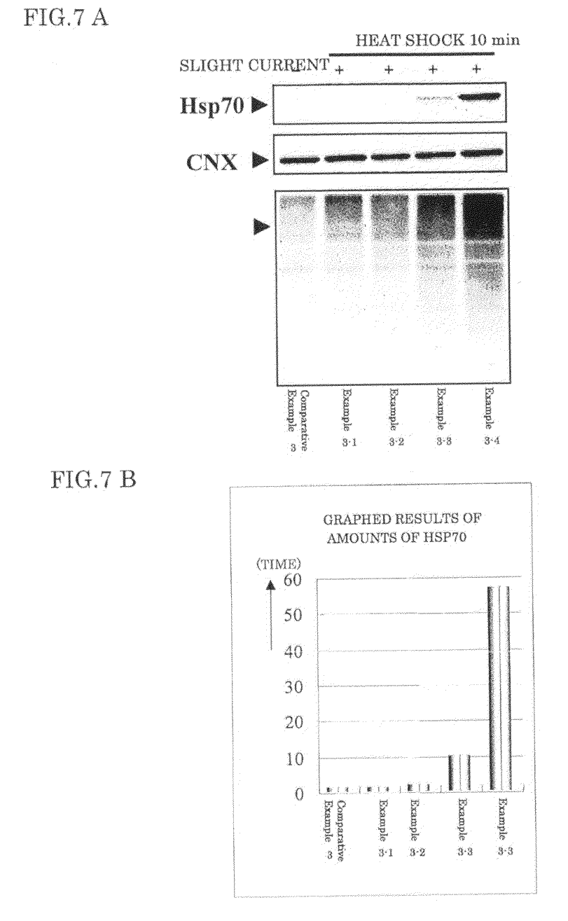

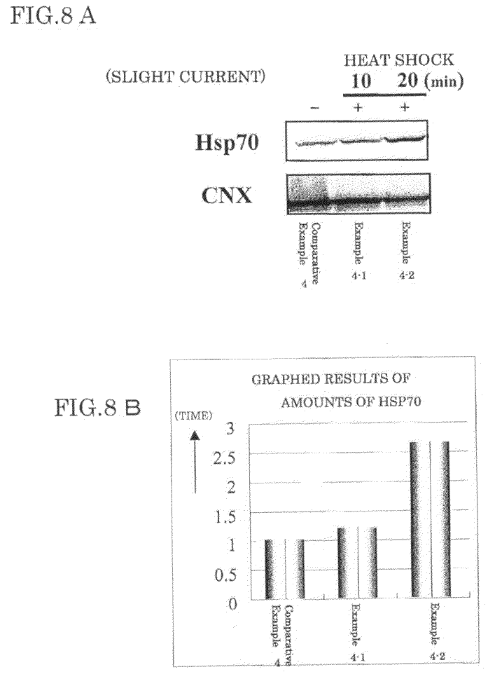

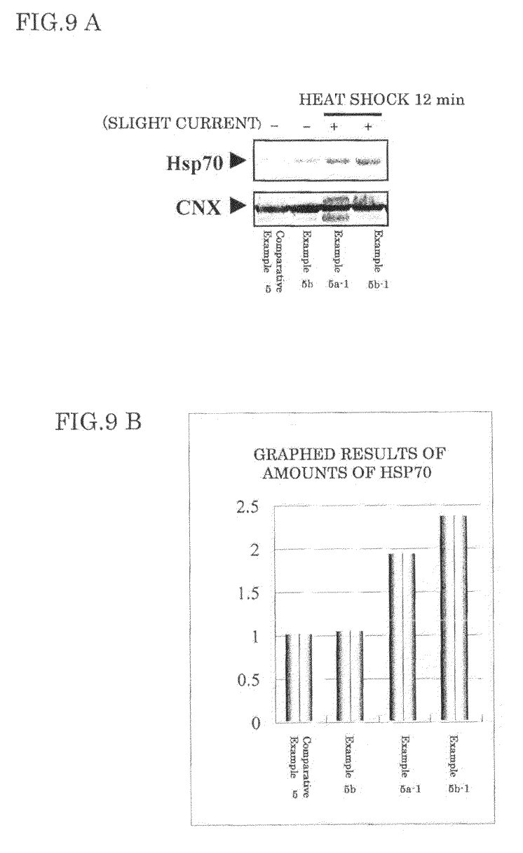

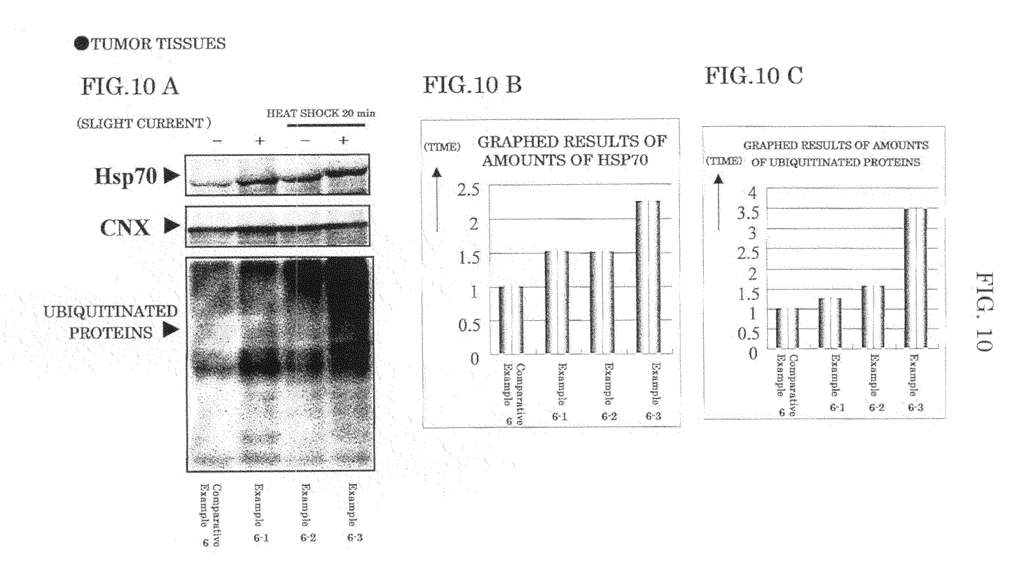

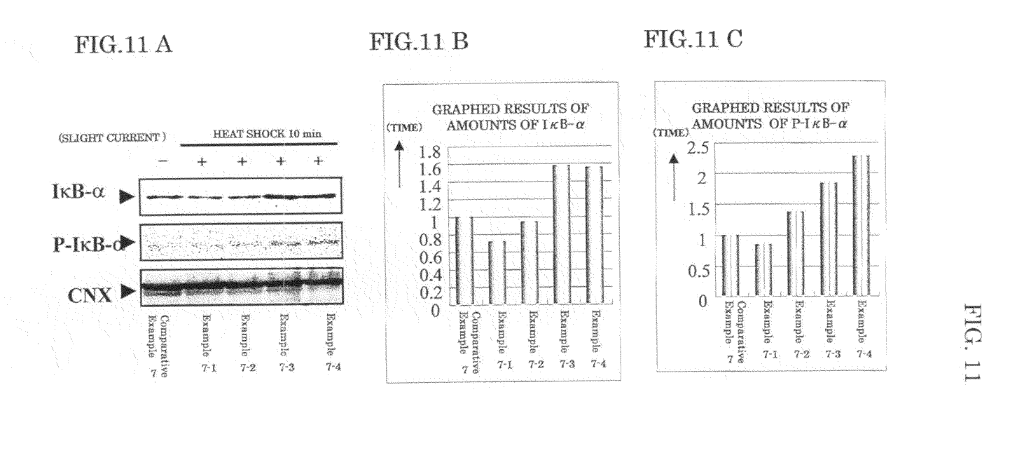

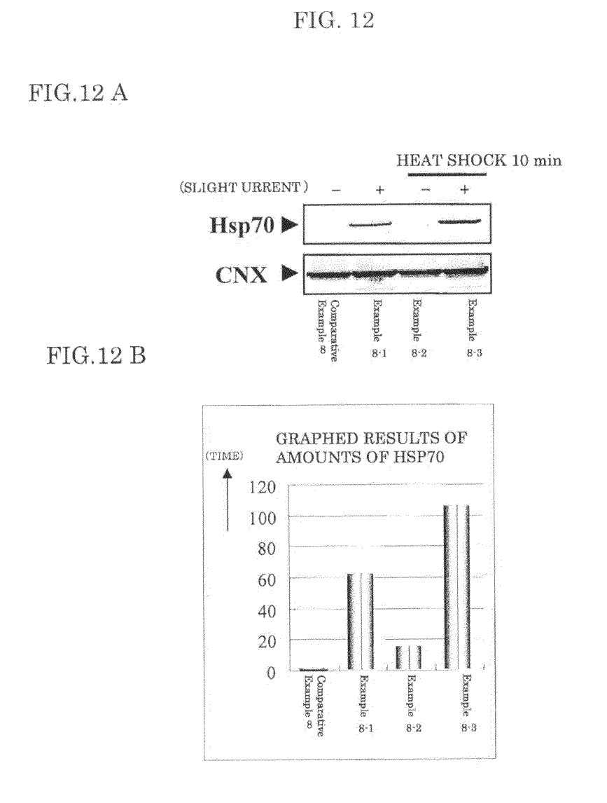

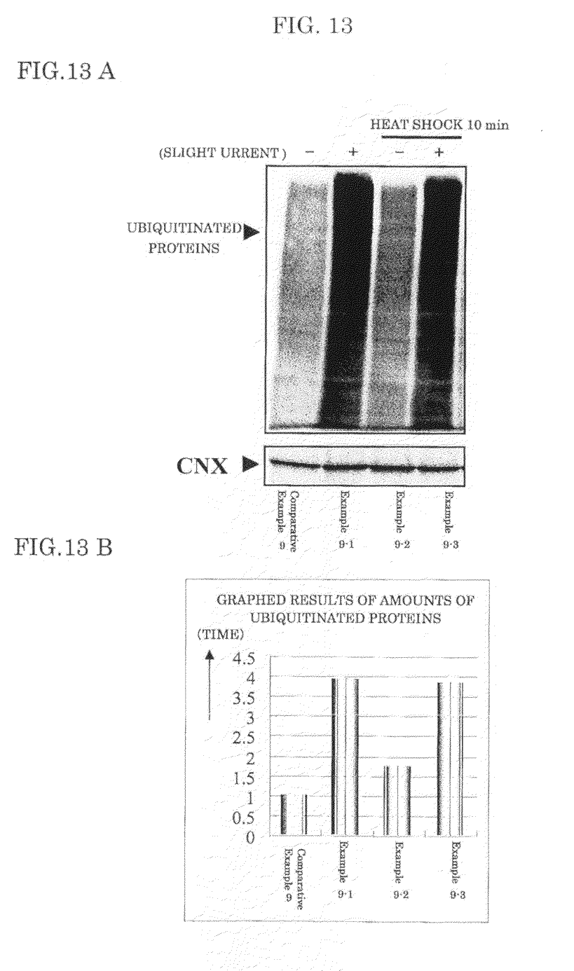

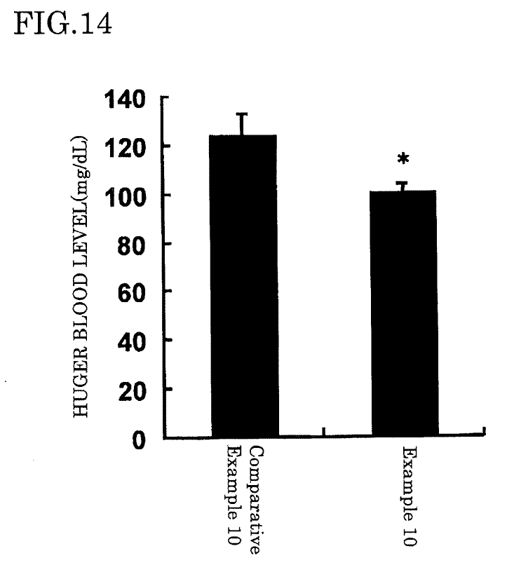

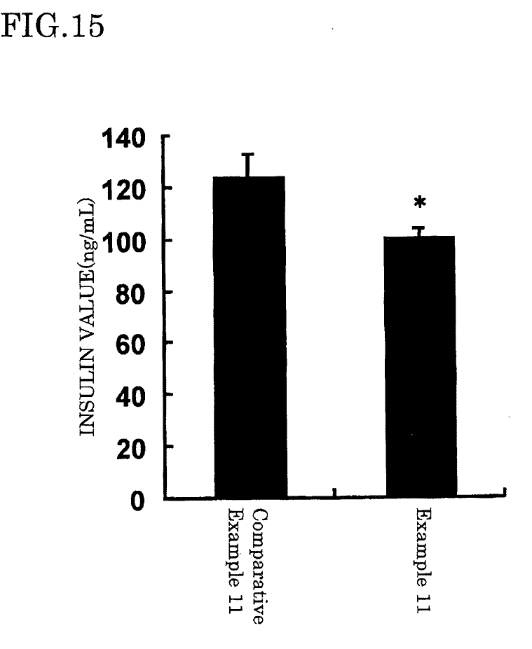

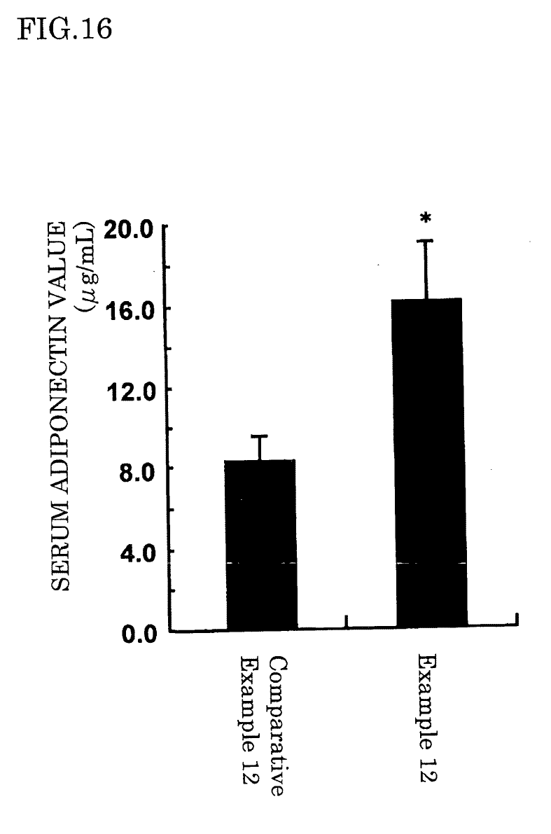

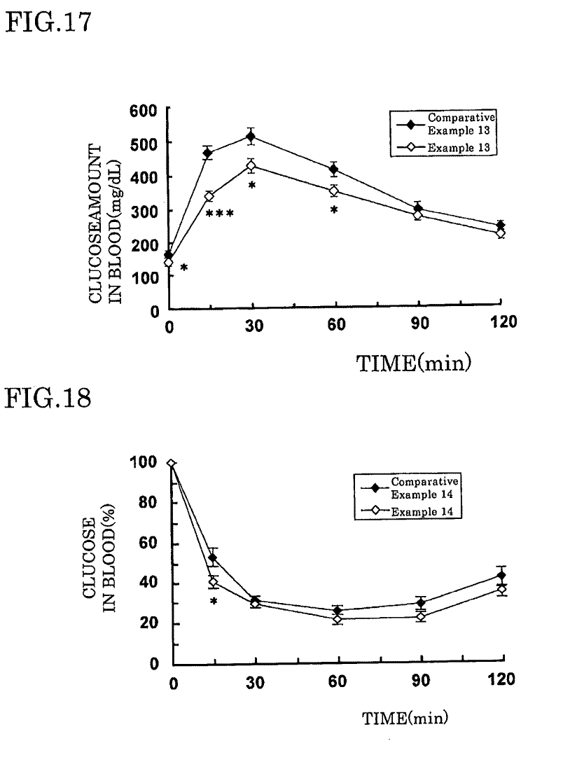

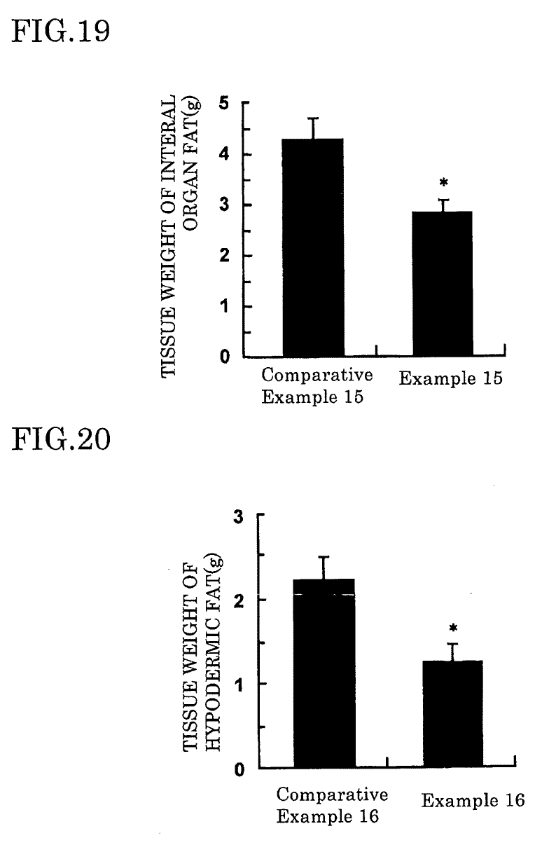

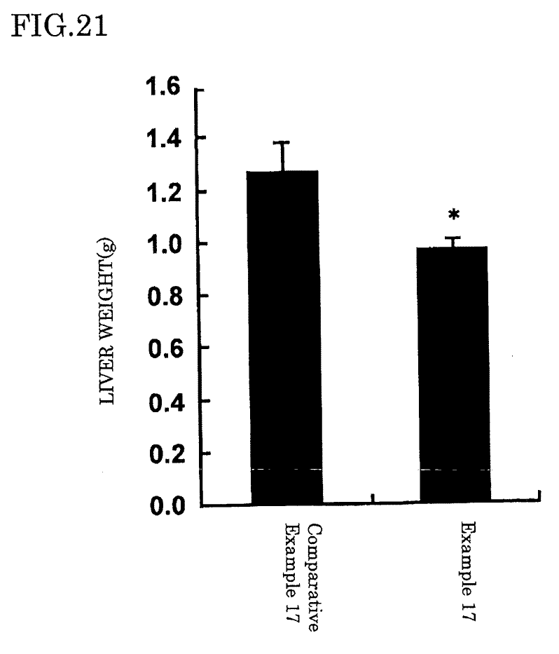

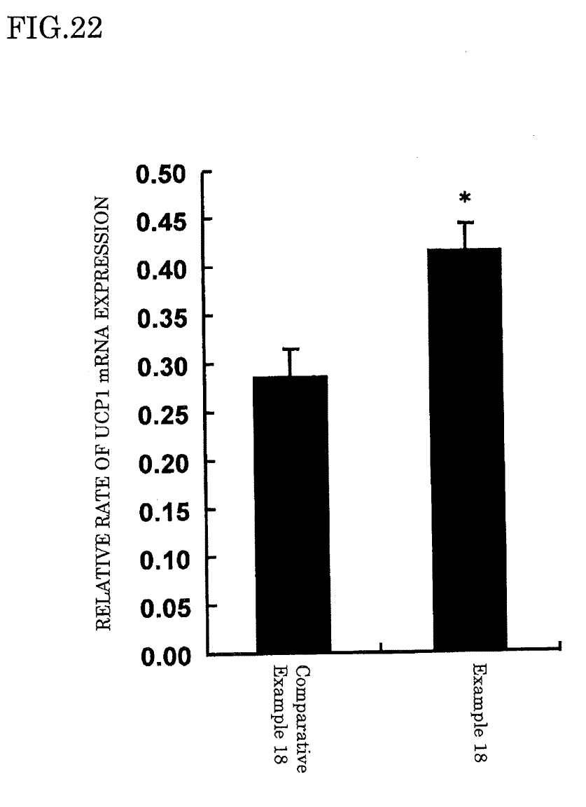

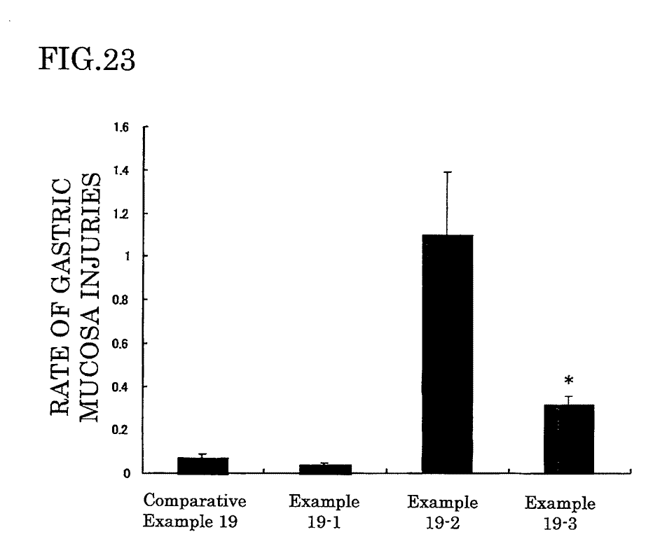

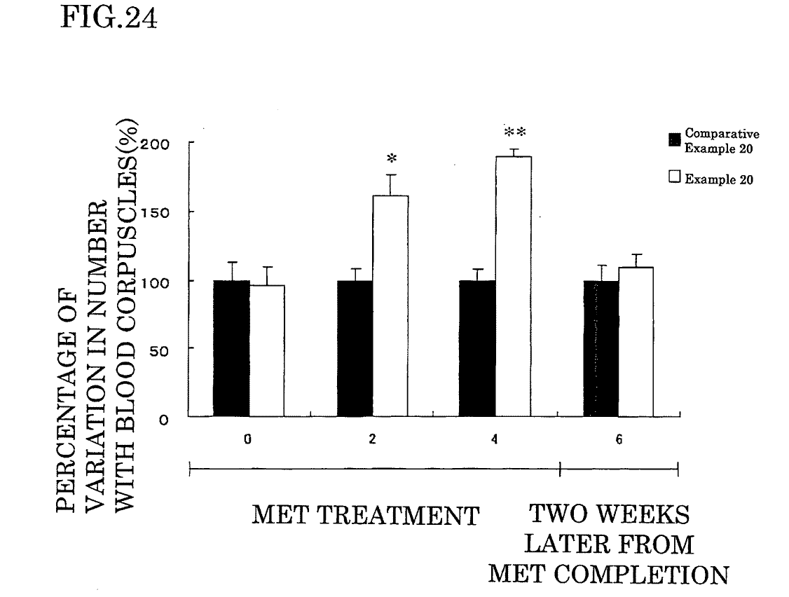

### BRIEF EXPLANATIONS OF DRAWINGS

[FIG. 1] is a general schematic configuration view of a therapeutic device according to one embodiment of the present invention.

[FIG. 2] is a plan view of a pad element of the therapeutic device of FIG. 1.

[FIG. 3] is a cross-sectional view of the pad element taken along the A-A line of FIG. 2.

[FIG. 4] is a cross-sectional view of the pad element taken along the B-B line of FIG. 3.

[FIGS. 5A-B] is the estimation results concerning the induction of HSP70 in the human's culture cell types.

[FIG. 6] is the estimation results concerning whether or not there are the injured cells in the human's culture cell types.

[FIGS. 7A-B] is the estimation results concerning the facilitation of the ubiquitination in the human's culture cell types.

[FIGS. 8A-B] shows the estimation results concerning the induction of HSP70 in the normal tissues of the nude mice with the one-time treatment.

[FIGS. 9A-B] shows the estimation results concerning the induction of HSP70 in the tumor tissues of the nude mice with the one-time treatment.

[FIGS. 10A-C] shows the estimation results concerning the facilitation of the ubiquitination in the tumor tissues of the nude mice with the one-time treatment.

[FIGS. 11A-C] shows the estimation results concerning the expression of IκB-α and the phosphorylated substance thereof in the nude mice with the one-time treatment in day and with the one-time treatment per day over the three days.

[FIGS. 12A-B] shows the estimation results concerning the induction of HSP70 in the culture cell types in which the genetic mutant cell ΔF508CFTR are stably and substantially expressed.

[FIGS. 13A-B] shows the estimation results concerning the facilitation of the ubiquitination in the culture cell types in which the genetic mutant cell ΔF508CFTR were stably and substantially expressed.

[FIG. 14] shows the fasting blood sugar level after the treatment which was carried out over 10 weeks with respect to the 2 type diabetic mouse models (high fat diet loaded mice).

[FIG. 15] shows the insulin value after the treatment which was carried out over 10 weeks with respect to the 2 type diabetic mouse models.

[FIG. 16] shows the serum adiponectin value after the treatment which was carried out over 10 weeks with respect to the 2 type diabetic mouse models.

[FIG. 17] shows the results of the glucose tolerance test which was carried out with respect to the 2 type diabetic mouse models.

[FIG. 18] shows the results of the insulin tolerance test which was carried out with respect to the 2 type diabetic mouse models.

[FIG. 19] shows the tissue weight of the internal organ fat after the treatment which was carried out over 10 weeks with respect to the 2 type diabetic mouse models.

[FIG. 20] shows the tissue weight of the hypodermic fat after the treatment which was carried out over 10 weeks with respect to the 2 type diabetic mouse models.

[FIG. 21] shows the liver weight after the treatment which was carried out over 10 weeks with respect to the 2 type diabetic mouse models.

[FIG. 22] shows the induction of UCP1 mRNA after the treatment which was carried out over 10 weeks with respect to the 2 type diabetic mouse models.

[FIG. 23] shows the rate of gastric mucosa injuries after the treatment which was carried out over 2 weeks with respect to the 2 type diabetic mouse models.

[FIG. 24] shows the percentage of variation in the number of white blood corpuscles after the treatment which was carried out over 2 weeks with respect to the 2 type diabetic mouse models.

### EXPLANATION OF REFERENCES

1, 2 Pad Element
3 Current Control Means
11, 21 Electrically Conductive Layer
12, 22 Electrically Insulative Layer
13, 23 Heating Layer
13a·13b, 23a·23b Electrodes
16 Carbon Fibers
14, 24 Covering Layer
100 Living Body
200 Electrical Power Source

### THE BEST MODE FOR EMBODYING THE INVENTION

A therapeutic device according to one embodiment of the present invention is explained together with a living-tissue normalization method based on FIGS. 1 to 3 below. FIG. 1 is a general schematic configuration view of a therapeutic device according to the present embodiment; FIG. 2 is a plan view of a pad element of the therapeutic device of FIG. 1; FIG. 3 is a cross-sectional view of the pad element taken along the A-A line of FIG. 2; and FIG. 4 is a cross-sectional view of the pad element taken along the B-B line of FIG. 3.

In each of the aforesaid drawings, the therapeutic device according to the present embodiment has an arrangement wherein a pair of pad elements 1 and 2 are adhered to different parts of a living body 100, and an electrical current is flowed between the pair of pad elements 1 and 2 from an electric power source 200 through a current control means 3 to thereby cure the living body 100. Each of the pad elements 1 and 2 includes an electrically conductive layer 11 or 21 adhered to the surface of the living body 100 and comprising an electrically conductive sheet, an electrically insulative layer 12 or 22 provided on a rear face of the electrically conductive layer 11 or 21 and comprising an electrically insulative sheet exhibiting a thermally conductive characteristic, a heating layer 13 or 23 provided on a rear face of the electrically insulative layer 12 or 22 and comprising a pair of electrodes 13a and 13b or 23a and 23b arranged at the side ends thereof and a resistance 13c or 23c provided between the pair of electrodes 13a and 13b or 23a and 23b, and a covering layer 14 or 24 provided on a rear face of the heating layer 13 or 23 and comprising an electrically insulative sheet exhibiting a thermally insulative characteristic. The current control means 3 is arranged so that the electrically conductive layers 11 and 21 of the pair of pad elements 1 and 2 are intermittently supplied with a slight direct current at predetermined regular intervals, and so that the pair of electrodes 13a and 13b and the pair of electrodes 23a and 23b are supplied with a direct current.

In the heating layer 13 or 23, each of the electrodes 13a and 13b or 23a and 23b is shaped as a strip-like electrode, and the resistance 13c or 23c is formed of a plurality of carbon fibers 16 extending in parallel to the pair of electrodes 13a and 13b or 23a and 23b therebetween, with a heating temperature of the heating layer 13 or 23 being 42° C.

The aforesaid current control means 3 supplies the electrically conductive layers 11 and 21 with the slight current at regular intervals of 55 Hz so that the electrically conductive layers 11 and 21 are intermittently turned ON over a time period from at least 10 min. to at most 30 min., with an applied voltage falling within a range from at least 0.03 V to at most 0.2 V.

With respect to slight currents, applied voltages, intermittent frequencies with which the aforesaid current control means 3 supplies living bodies or living tissues, explanation is made based on human's test results. In a human's test, a voltage falling within a range from 0.2 [V] to 0.4 [V] was applied to the legs of a human. On the presumption that the human's resistance was about 0.2 [MΩ] (a resistance in the oral cavity), a voltage drop (potential difference) falling within a range from 0.1 [V] to 0.2 [V] might be caused in the human's body. With the potential difference from 0.1 [V] to 0.2 [V] and the human's resistance of about 0.2 [MΩ], it was presumed that the aforesaid current control means 3 supplied the human with the slight current falling within a range from 0.1 [V] to 0.2 [V].

In this human's test, the voltage of 0.3 [V] was applied to the legs of the tested human, and the intermittent frequency was varied between 35 [Hz] and 150 [Hz]. Feelings (comfortable feelings or uncomfortable feelings) of the tested human were researched as follows:

First, at 35 [Hz], it was found that the tested human still had a strange feeling, and that he had a bad feeling while being subjected to the treatment over a long time. At 45 [Hz], it was found that the tested human had not only a strong numb feeling and but also a bad (uncomfortable) feeling, and that it was hard for the tested human to be subjected to the treatment over a long time. At 50 [Hz], it was found that the tested human had a comfortable feeling falling within an acceptable range. At 55 [Hz], it was found that the tested human had a vary comfortable feeling. At 60 [Hz], it was found that the tested human had a comfortable feeling falling within the acceptable range.

Also, at 65 [Hz], stimuli became somewhat smaller. When the applied voltage was increased to 0.350 V, it was found that the tested human felt similar stimuli to those in the case of 55 [Hz], but he had not a comfortable feeling. At 70 [Hz], it was found that the tested human hardly felt stimuli. At 75 [Hz], the tested human hardly felt stimuli. When the applied voltage was increased to 0.400 V, it was found that the tested human felt few stimuli. At 100 [Hz], the tested human felt no stimuli. Although the applied voltage was increased to 0.600 V, it was found that the tested human felt no stimuli.

Also, in cases where voltages of 0.25 [V], 0.3 [V], 0.35 [V] and 0.4 [V] were applied, optimum frequencies, at which these direct voltages were intermittently applied, were researched by a test. When the applied voltage was 0.250 [V], the frequency was varied within a range 35<Hz<50. When the frequency was less than 35 [Hz], the tested human had not only a strong numb feeling but also a bad feeling. When the frequency was more than 50 [Hz], the tested human had no feeling. Also, when the applied voltage was 0.300 [V], the frequency was varied within a range 45<Hz<60. When the frequency was less than 45 [Hz], the tested human had not only a strong numb feeling but also a bad feeling. When the frequency was more than 75 [Hz], the tested human had no feeling. Further, when the applied voltage was 0.400 [V], the frequency was varied within a range 65<Hz<75. When the frequency was less than 75 [Hz], the tested human's muscle was strongly contracted or the tested human had a bad feeling. When the frequency was more than 75 [Hz], the tested human had no feeling.

From the aforesaid test results, it is found that the optimum frequency is 55(±1) [Hz] at the applied voltage of 0.3 [V], and that it is desirable to carry out a control by the current control means 3 within the range from 50 [Hz] to 60 [Hz].

Also, due to the function of electrical signals to a living body or living tissue, it is possible to control a slight current by the above-mentioned current control means 3 as follows. Namely, a slight current corresponding to a bioelectric current is forcibly flowed into non-excitable cells of the living body or living tissue to thereby activate the cells through proteins in the body. Like this, the slight current corresponding to the bioelectric current is flowed into only the non-excitable cells, but the external current corresponding to the bioelectric current cannot be flowed into excitable cells such as muscular cells and so on, and thus stimuli such as contraction and so on cannot be given to the excitable cells.

Accordingly, the current control means 3 controls the flowing of the slight current at a current level so that the excitable cells, for example, the muscular cells are not subjected to uncomfortable contraction.

Next, an induction effect of proteins expressed by an operation of the therapeutic device according to the present embodiment will be explained based on the following tests in connection with a human culture cell type, a nude mouse, and a culture cell type in which ΔF508CFTR was stably and substantially expressed.

### EXAMPLE

### Study Items

(1) Estimation concerning human culture cell types was carried out with respect to the following three Items i), ii) and iii):
i) Induction of HSP70
ii) Injured Cells or Not
iii) Facilitation of Ubiquination

(2) Estimation of nude mice was carried out with respect to the following four Items i), ii), iii) and iv):
i) Induction of HSP70 in Normal Tissue with One-time Treatment
ii) Induction of HSP70 in Tumor Tissue with One-time Treatment
iii) Facilitation of ubiquination in Tumor Tissue
iv) Expression of IκB-α and Phosphorylated Substance Thereof in Normal Tissue with One-time Treatment in Day and with One-time Treatment per Day Over three Days

(3) Estimation concerning the ΔF508CFTR stably/substantially expressed culture cell type was carried out with respect to the following two Items i) and ii):
i) Induction of HSP70
ii) Facilitation of Ubiquination

(4) Estimation concerning 2 type diabetic nude mice (high fat diet loaded mice) was carried out with respect to the following nine Items i), ii), iii), iv), v), vi), vii), viii) and ix): Note that the following analyses were made after ten weeks in each of which a treatment based on the living-tissue normalization method of the present invention was carried out twice.
i) Fasting Blood Sugar Level
ii) Insulin Value
iii) Serum Aadiponectin Value
iv) Glucose Tolerance Test
v) Insulin Tolerance Test
vi) Tissue Weight of Internal Organ Fat
vii) Tissue Weight of Hypodermic Fat
viii) Liver Weight
ix) Induction of UCP1 mRNA in Brawn Fat Cells

(5) Estimation concerning mouse models having an acute gastric ulcer was carried out with respect to the following Item: Note that the following analysis was made after two weeks in each of which a treatment based on the living-tissue normalization method of the present invention was carried out twice.
i) Rate of Gastric Mucosa Injuries

(6) Estimation concerning normal mice was carried out with respect to the following Item: Note that the following analysis was made every two weeks in each of which the treatment of the present invention was carried out once, with the first treatment being carried out before two weeks from the first corpuscle measurement.
i) Number of White Blood Corpuscles Per 1 mL Blood

### (Methods for Examples)

1) In measurement of HSP70, an immunoblotting method was carried out by using mouse-anti HSP70 monoclonal antibodies, and the combined antibodies were detected and measured by using the western blot detection kit (manufactured by Amersham Inc.) based on an enhanced chemiluminescence (ECL) method. As loading controls, calnexin (CNX) was detected.

2) In measurement of ubiquitinated proteins, the immunoblotting method was carried out by using the mouse-anti HSP70 monoclonal antibodies, and the combined antibodies were detected and measured by using the western blot detection kit (manufactured by Amersham Inc.) based on the enhanced chemiluminescence (ECL) method.

3) In measurement of IκB-α and IκB-α phosphorylated substance, the immunoblotting method was carried out by using rabbit-anti IκB-a polyclonal antibodies and rabbit-anti phosphorylated IκB-α polyclonal antibodies, and the combined antibodies were detected and measured by using the western blot detection kit (manufactured by Amersham Inc.) based on the enhanced chemiluminescence (ECL) method.

4) By photographing figure transformation, using the handstand type stereo microscope (manufactured by Olympus Inc.), it was confirmed whether or not there were injured cells derived from a treatment based on the therapeutic device.

5) A blood sugar level was measured by using the self-type blood sugar determination tool (manufactured by Roches Inc.).

6) An insulin value and a serum adiponection value were measured by using Lipo SEARCH (the unique sensitive gel filtration HPLC system) of Skylight Biotech Inc.

7) UCP1 mRNA was detected and measured by using the RT-PCR kit (manufactured by Takara Inc.) for carrying an RT-PCR.

8) Mouse models having an acute gastric ulcer were produced by orally giving hydrochloride ethanol to them. To determine a rate of gastric mucosa injuries, an are of injured gastric mucosa were measured by using an anatomy microscope, and the rate of gastric mucosa injuries was defined by the following formula:

Rate of Gastric Mucosa Injuries=(Area of Injured Gastric Mucosa/Total Gastric Mucosa Area)×10

9) A number of white blood corpuscles was measured by using the corpuscle measurement device SysmexF-520 (manufactured by Sysmex Inc.).

(Results of Examples)

The following estimation results concerning the human culture cell types were obtained by the aforesaid method, as shown in FIGS. 5, 6 and 7:

i) Induction of HSP70 (see: FIGS. 5(A) and 5(B))

A part of the samples which was subjected to only a slight current treatment over the time period of 10 min. (Example 1-1), another part of the samples which was subjected to only a thermal treatment over the time period of 10 min. (Example 1-2), and still another part of the samples which was simultaneously subjected to both a slight current treatment and a thermal treatment over the time period of 10 min. (Example 1-3) were statically placed and cultured over the time period of 5 hr., and then induction levels of HSP70 in these samples were detected by using the western blot technique. As a result, in the samples which were subjected to only the slight current treatment (Example 1-1) and the samples which were subjected to only the thermal treatment (Example 1-2), it was found that the respective induction levels of HSP70 were about 3.5 times and about 1.9 times (see: FIG. 5(B)). Especially, in the samples (Example 1-3) which were simultaneously subjected to both the slight current and thermal treatments, it was found that the induction level of HSP70 was remarkably about 5.2 times.

ii) Injured Cells or Not (see: FIG. 6)

Under the aforesaid conditions, it was examined whether or not there were injured cells by using the electron microscope. As a result, it was confirmed that no cells were injured in a part of the samples which was subjected to only a slight current treatment (Example 2-1), another part of the samples which was subjected to only a thermal treatment (Example 2-2), and still another part of the samples which was simultaneously subjected to both a slight current treatment and a thermal treatment (Example 2-3).

iii) Facilitation of Ubiquitination (see: FIGS. 7(A) and 7(B))

As a treatment condition, samples were simultaneously subjected to both a slight current treatment and a thermal treatment over the time period of 10 min., as stated referring to FIG. 5. Then, a part of the samples (Example 3-1), another part of the samples (Example 3-2), still another part of the samples (Example 3-3) and still yet another part of the samples (Example 3-4) were statically placed and cultured over time periods of 0, 2, 5 and 8 hr., respectively. Subsequently, respective amounts of ubiquitinated proteins in these samples were detected by using the western blot technique. As a result, it was confirmed that the ubiquitination was exponentially facilitated 10 times in the culture over 5 hr. and 68 times in the culture over 8 hr.

The following estimation results concerning the nude mice were obtained by the aforesaid method, as shown in FIGS. 8, 9, 10 and 11:

i) Induction of HSP70 in Normal Tissues with One-time Treatment (see: FIGS. 8(A) and 8(B))

As a treatment condition, normal tissues of a part of the nude mice were simultaneously subjected to both a slight current treatment and a thermal treatment over the time period of 10 min. (Example 4-1), and normal tissues of another part of the nude mice were simultaneously subjected to both a slight current treatment and a thermal treatment over the time period of 20 min. (Example 4-2). Then, these nude mice were kept over the time period of 6 hr., and then induction levels of HSP70 in the normal tissues (e.g. large intestines) were detected by using the western blot technique. As a result, in the treatment over 20 min. (Example 4-2), it was found that the induction level of HSP70 was remarkably about 2.7 times.

ii) Induction of HSP70 in Tumor Tissues with One-time Treatment (see: FIGS. 9(A) and 9(B))

As a treatment condition, tumor tissues of the nude mice were simultaneously subjected to both a slight current treatment and a thermal treatment over the time period of 20 min., and these nude mice were kept over the time period of 6 hr. Then, the induction levels of HSP70 in the tumor tissues were detected by using the western blot technique, as shown in Examples 5a-1 and 5b-1. As a result, in even the tumor tissues, it was found that the induction level of HSP70 was remarkably about 1.9 times and 2.4 times.

iii) Facilitation of Ubiquitination in Tumor Tissues (see: FIGS. 10(A), 10(B) and 10(C))

Tumor tissues of a part of the mode mice were subjected to only a slight current treatment over the time period of 20 min. (Example 6-1), tumor tissues of another part of the nude mice were subjected to only a thermal treatment over the time period of 20 min. (Example 6-2), and tumor tissues of still another part of the nude mice were simultaneously subjected to both a slight current treatment and a thermal treatment over the time period of 30 min. (Example 6-3). Then, these nude mice were kept over the time period of 6 hr., and amounts of the ubiquitinated proteins in the tumor tissues were detected by using the western blot technique. As a result, in the nude mice which were subjected to only the slight current treatment (Example 6-1) and the nude mice which were subjected to only the thermal treatment (Example 6-2), it was found that the induction level of HSP70 was 1.5 times, and that the increases in the ubiquitinated proteins was 1.3 times and 1.6 times in the respective cases. Especially, in the nude mice which were simultaneously subjected to both the slight current and thermal treatments (Example 6-3), it was found that the induction level of HSP70 was remarkably about 2.3 times, and that the increase in the ubiquitinated proteins was remarkably 3.5 times.

iv) Expression of IκB-α and Phosphorylated Substance Thereof in Normal Tissue with One-time Treatment in Day and with One-time Treatment per Day Over three Days (see: FIGS. 11(A), 11(B) and 11(C))

Normal tissues of a part of the nude mice were simultaneously subjected to both a slight current treatment and a thermal treatment one time (Examples 7-1 and 7-2), and normal tissues of another part of the nude mice were simultaneously subjected to both a slight current treatment and a thermal treatment one time a day over three days (Examples 7-3 and 7-4). Then, IκB-α and IκB-α phosphorylated substance were detected in the normal tissues of these node mice by using the western blot technique. As a result, in the nude mice which were simultaneously subjected to the slight current and thermal treatments one time a day over three days (Examples 7-3 and 7-4), it was found that amounts of IκB-α and IκB-α phosphorylated substance were remarkably increased in comparison with the Comparative Example.

(3) The following estimation results concerning the ΔF508CFTR stably/substantially expressed culture cell types were obtained by the aforesaid method, as shown in FIGS. 11 and 12:

i) Induction of HSP70 (see: FIGS. 12(A) and 12(B))

A part of the samples which was subjected to only a slight current treatment over the time period of 10 min. (Example 8-1), another part of the samples which was subjected to only a thermal treatment over the time period of 10 min. (Example 8-2), and still another part of the samples which was simultaneously subjected to both a slight current treatment and a thermal treatment over the time period of 10 min. (Example 8-3) were statically placed and cultured over the time period of 5 hr., and then induction levels of HSP70 in these samples were detected by using the western blot technique. As a result, in the samples which were subjected to only the slight current treatment (Example 8-1) and the samples which were subjected to only the thermal treatment (Example 8-2), it was found that the respective induction levels of HSP70 were 62 times and 18 times. Nevertheless, in the samples which were simultaneously subjected to both the slight current and thermal treatments (Example 8-3), it was found that the induction level of HSP70 was remarkably about 105 times.

ii) Facilitation of Ubiquination (see: FIGS. 13(A) and 13(B))

A part of the samples which was subjected to only a slight current treatment over the time period of 10 min. (Example 9-1), another part of the samples which was subjected to only a thermal treatment over the time period of 10 min. (Example 9-2), and still another part of the samples which was simultaneously subjected to both a slight current treatment and a thermal treatment over the time period of 10 min. (Example 9-3) were statically placed and cultured over the time period of 5 hr., and then ubiquitinated ΔF508CFTR in these samples were detected by using the western blot technique. As a result, in the samples which were subjected to only the slight current treatment (Example 9-1) and the samples which were simultaneously subjected to both the slight current and thermal treatments (Example 8-3), it was found that the detected ubiquitination of ΔF508CFTR in these cases was about 3.9, resulting in facilitation of the ubiquitination.

(4) The following estimation results concerning the 2 type diabetic nude mice (high fat diet loaded mice) were obtained by the aforesaid method, as shown in FIGS. 14, 15, 16, 17, 18, 19, 20, 21 and 22:

i) Fasting Blood Sugar Level After Treatment over 10 Weeks (see: FIG. 14)

In a group of high fat diet loaded mice which was simultaneously subjected to both a light current treatment and a thermal treatment (Example 10), a significant decrease in the fasting blood sugar level was confirmed (P<0.05, and n=8).

ii) Insulin Value After Treatment over 10 Weeks (see: FIG. 15)

In a group of high fat diet loaded mice which was simultaneously subjected to both a light current treatment and a thermal treatment (Example 11), a significant decrease in the insulin value was confirmed (P<0.05, and n=8).

iii) Serum Adiponectin Value After Treatment over 10 Weeks (see: FIG. 16)

In a group of high fat diet loaded mice which was simultaneously subjected to both a light current treatment and a thermal treatment (Example 12), a significant increase in the serum adiponectin value was confirmed (P<0.05, and n=8).

iv) Glucose Tolerance Test (see: FIG. 17)

In a group of high fat diet loaded mice which was simultaneously subjected to both a light current treatment and a thermal treatment (Example 13), an improvement in a glucose tolerance ability was significantly confirmed (P<0.001, and n=8).

v) Insulin Tolerance Test (see: FIG. 18)

In a group of high fat diet loaded mice which was simultaneously subjected to both a light current treatment and a thermal treatment (Example 14), an improvement in an insulin sensitivity was significantly confirmed (P<0.05, and n=8).

vi) Tissue Weight of Internal Organ Fat After Treatment over 10 Weeks (see: FIG. 19)

In a group of high fat diet loaded mice which was simultaneously subjected to both a light current treatment and a thermal treatment (Example 15), it was confirmed that a tissue weight of the internal organ fat was significantly reduced (P<0.05, and n=8).

vii) Tissue Weight of Hypodermic Fat After Treatment over 10 Weeks (see: FIG. 20)

In a group of high fat diet loaded mice which was simultaneously subjected to both a light current treatment and a thermal treatment (Example 16), it was confirmed that a tissue weight of the hypodermic fat was significantly reduced (P<0.05, and n=8).

viii) Liver Weight After Treatment over 10 Weeks (see: FIG. 21)

In a group of high fat diet loaded mice which was simultaneously subjected to both a light current treatment and a thermal treatment (Example 17), it was confirmed that a tissue weight of the liver weight was significantly reduced (P<0.05, and n=8).

ix) Induction of UCP1 mRNA After Treatment over 10 Weeks (see: FIG. 22)

In a group of high fat diet loaded mice which was simultaneously subjected to both a light current treatment and a thermal treatment (Example 18), it was confirmed that an amount of UCP1 mRNA expressed in the brawn fat cells was significantly increased (P<0.05, and n=8).

(5) The following estimation results concerning the mouse models having the acute gastric ulcer were obtained by the aforesaid method, as shown in FIG. 23:

i) Rate of Gastric Mucosa Injuries After the treatment over 2 Weeks (see: FIG. 23)

In a group of mouse models which was simultaneously subjected to both a light current treatment and a thermal treatment (Example 19-1), another group of mouse models having the acute gastric ulcer (Example 19-2), and still another group of mouse models having the acute gastric ulcer, which was simultaneously subjected to both a light current treatment and a thermal treatment (Example 19-3), rates of the gastric mucosa injuries were measured. As a result, in the group of mouse models having the acute gastric ulcer, which was simultaneously subjected to both a light current treatment and a thermal treatment (Example 19-3), a significant decrease in the rate of the gastric mucosa injuries was confirmed (P<0.05, and n=4·7).

(6) The following estimation results concerning the normal mice were obtained by the aforesaid method, as shown in FIG. 24:

i) Percentage of Variation in Number of White Blood Corpuscles (see: FIG. 24)

In a group of normal mice which was simultaneously subjected to both a light current treatment and a thermal treatment (Example 20), it was confirmed that a number of white blood corpuscles per the blood of 1 mL was significantly increased (P<0.01, and n=6).

As is apparent from the foregoing, the living-tissue normalization method and the therapeutic device according to the present invention feature a very superior HSP induction ability, and are effective against a variety of diseases. Also, the living-tissue normalization method and the therapeutic device according to the present invention feature high safety, and thus it can be expected that they are of clinically superior utility. As examples of the aforesaid variety of diseases, there are cranial nerve diseases, heart systema vasorum diseases, digestive systema diseases, metabolism diseases, autoimmune diseases, degenerative diseases, ischemia neuropathy, ischemia/reperfusion injuries, cystic fibrosis, malignant tumors, infection diseases, liver failures, renal failures, drug poisoning, heavy metal poisoning, radiation poisoning, ultraviolet hazard, living body invasions, aging and so on. As the cranial nerve diseases, there are apoplexy, apoplexy aftereffects, late onset nerve cell deaths, Alzheimer's diseases, Parkinson's diseases, multiple scleroses, Creutzfeldt-Jakob's diseases and so on.

Further, the living-tissue normalization method and the therapeutic device according to the present invention activate a normalization mechanism of a living body or a living tissue through the intermediary of ubiquitinated proteins. About 80% of proteins in cells are ubiquitinated, and then the ubiquitinated proteins are resolved by proteasome. However, when a function of the proteasome is inhibited, an amount of ubiquitinated proteins which are not resolved is increased in the cells, and thus the cells select a way to the programmed cell death. At present, a proteasome inhibitor based on this principle is noticed as an anticancer drug (Julian A. Cancer Cell, 2003, and Angelika M. B et al. European Journal of Cancer, 2004).

The proteasome inhibitor inclines to acting on cells in a growth phase in which synthesis and resolution of proteins are actively carried out. Since a control malfunction of proteins concerning cell growth is caused in tumor cells, a rate of cell growth is very larger in comparison with normal cells. Thus, tumor tissues are susceptible to influence of the proteasome inhibitor acting on the cells in the growth phase. The living-tissue normalization method and the therapeutic device according to the present invention contributes to facilitation of ubiquitination so that an amount of ubiquitinated proteins is considerably increased in cells. Thus, the proteasome is brought to a saturated-state, and this saturated-state is similar to a state in which the function of the proteasome is inhibited.

In particular, the living-tissue normalization method and the therapeutic device according to the present invention also feature an antitumoral effect based on the inhibition of the proteasome. Also, on the basis of these principles, the antitumoral effect can be expected to be exercised to the tumor cells. As diseases which can be improved by the normalization mechanism of the ubiquitin-proteasome system based on the ubiquitinated proteins, there are neuro-degeneration diseases (for example, Parkinson's diseases, Alzheimer's diseases, amyotrophic lateral scleroses (ALS), myoclonus epilepsy and so on), cancer diseases (for example, family breast cancers, ovary cancers and so on), xerodema pigmentosum and so forth.

Since excess activation of NF-κB causes rheumatism, asthma, a variety of inflammatory diseases such as dermatitis and so on, autoimmune diseases, viral diseases, arterioscleroses and so forth, control of NF-κB is clinically very significant. Thus, in the therapeutic device according to the present invention, since an amount of NF-κB is increased while phosphorylation of NF-κB is suitably suppressed due to the effect of the slight current treatment and so on, there is an expectation of improving an variety of clinical conditions resulting from an excess immune response based on NF-κB.

In the above-mentioned embodiments, although the present invention are explained in connection with tissue cells of animals, such as human culture cell types, nude mice, culture cell types in which ΔF508CFTR is stably and substantially expressed, the present invention may be applied to vegetable tissue cells to obtain similar advantages.

1. A living-tissue normalization method characterized by intermittently flowing a slight direct current through a living body or living tissue at predetermined regular intervals to thereby activate a normalization mechanism of said living body or living tissue through the intermediary of protein.

2. The living-tissue normalization method as set forth in claim 1, characterized by applying heat to said living body or living tissue through which said slight direct current is flowed.

3. The living-tissue normalization method as set forth in claim 1, characterized in that said protein comprises ubiquitinated protein.

4. The living-tissue normalization method as set forth in claim 1, characterized in that said protein comprises heat shock protein.

5. The living-tissue normalization method as set forth in claim 1, characterized in that said protein comprises I-κB protein.

6. The living-tissue normalization method as set forth in claim 1, characterized in that the intermittent intervals of said slight direct current falls within a range from 30 Hz to 100 Hz.

7. The living-tissue normalization method as set forth in claim 2, characterized in that a temperature of said heating falls within a range from 38° C. to 45° C.

8. A therapeutic device in which a pair of pad elements are adhered to different parts of a living body or living tissue, and an electrical current is flowed between the pair of pad elements through a current control means to thereby cure said living body,
characterized in that each of said pad elements includes an electrically conductive layer adhered to a surface of said living body or living tissue and comprising an electrically conductive sheet, an electrically insulative layer provided on a rear face of the electrically conductive layer and comprising an electrically insulative sheet exhibiting a thermally conductive characteristic, and a heating layer provided on a rear face of the electrically insulative layer and comprising a pair of electrodes arranged at the side ends thereof and a resistance provided between the pair of electrodes, and that said current control means is arranged so that the electrically conductive layers of the pair of pad elements are intermittently supplied with a slight direct current at predetermined regular intervals, and so that the pair of electrodes are supplied with a direct current.

9. The therapeutic device as set forth in claim 8, characterized in that each of the pair of electrodes is shaped as a strip-like electrode in the heating layer of said pad element, and that said resistance is formed of a plurality of carbon fibers extending in parallel to the pair of electrodes therebetween.

10. The therapeutic device as set forth in claim 8, characterized in that the direct current flowing into each of said electrically conductive layers is intermittently supplied at a frequency falling within a range from 50 Hz to 60 Hz.

11. The therapeutic device as set forth in claim 8, characterized in that the heating layer of each of said pair of pad elements is heated at a temperature falling within a range from 38° C. to 45° C.

12. The therapeutic device as set forth in claim 8, characterized in that the direct current flowing through said electrically conductive layer features a supply time of at least 1 min., and that said direct current has a voltage which is in reverse proportion to said supply time, and which is set within a range from 0.01 V to 0.4 V.
